(19) United States
(12) Patent Application Publication
(10) Pub. No.: US 2021/0338099 A1
(43) Pub. Date: Nov. 4, 2021

(54) Gastrointestinal motility measurement

(71) Applicant: Xiaoning Huai, Sunnyvale, CA (US)

(72) Inventor: Xiaoning Huai, Sunnyvale, CA (US)

(73) Assignee: Real Image Technology Co., Ltd, Shenzhen (CN)

(21) Appl. No.: 17/372,518

(22) Filed: Jul. 12, 2021

(51) Int. Cl. A61B 5/07; A61B 5/00

(52) U.S. Cl. CPC A61B 5/073; A61B 5/0077; A61B 2560/0209; A61B 5/42; A61B 2562/0223; A61B 5/6846

(57) ABSTRACT

The invention discloses a gastrointestinal motility measurement system based on a digestive tract capsule, which comprises a data acquisition module, a data processing module and a capsule. The data acquisition module is configured in the capsule, and comprises an ultrasonic distance measuring device or a 3D camera for acquiring a depth map or a point cloud of the inner wall of the digestive tract; The data processing module is used for processing the depth map or the point cloud to obtain the surface data of the inner wall surface of the digestive tract; Further extraction of morphological features, including the inner wall of the digestive tract, curvature, inner diameter and volume, can be used as a reference for the evaluation of gastrointestinal motility.

### TECHNICAL FIELD

The invention relates to the technical field of medical devices, in particular to gastrointestinal capsule.

### BACKGROUND

Gastrointestinal motility is important to human physiology and pathology. The measurement of gastrointestinal motility in the prior art is mainly based on the tracking of radioactive markers, as disclosed in U.S. patent application Ser. No. 15/881,671. Because radiation examination is harmful to organisms, the basic research and clinical application of gastrointestinal motility need a non-invasive testing scheme in vivo. Light, sound and magnetism are commonly used noninvasive testing vehicles. The 3D camera and gastrointestinal capsule robot with magnetic positioning have been commercially available, which provides a good technical feasibility for the scheme of the invention. The capsule robot can include sensors, controllers and intelligent processors. The sensor and at least part of the controller are usually located in the capsule, the intelligent processor is usually located in an external control terminal, and the sensor, the controller and the intelligent processor are usually connected by wired or wireless communication links.

### SUMMARY OF THE INVENTION

The invention provides a 3D ultrasonic capsule for measurement of morphologies of the digestive tract, and the capsule is configured to obtain the directional cavity diameter of the inner wall of the digestive tract using a plurality of pairs of reversely positioned ultrasonic ranging probes which form a spherical array of ultrasonic ranging probes. The endpoints of each directional cavity diameter are located on the opposite inner walls of the digestive tract and contains a pair of data of the depth map. The capsule contains a magnet for positioning the pose and position of the capsule, and the depth data of the inner wall of the digestive tract are collected in a target area in the digestive tract.

The invention provides a first method for measurement of gastrointestinal motility, comprising the following steps:

obtain the depth map or point cloud of the inner wall of digestive tract. The surface data of the inner wall of digestive tract were obtained. The morphological features of the surface data are extracted, and the morphological features include one or more of the anatomical parts of the inner wall of the digestive tract, curvature, inner diameter and volume.

The invention also provides a second method for measurement of gastrointestinal motility, comprising driving a capsule to a target area of digestive tract and applying an intervene magnetic force on the capsule by a magnetron;

obtaining data of a first transit time of the capsule when no magnetic force is applied on the capsule;
obtaining data of a second transit time of the capsule when a first magnitude of a magnetic force is applied on the capsule and the difference between the first transit time and the second transit time is bigger than a threshold;
obtaining data of a second set of transit time of the capsule when the magnetic force is increased from the first magnitude to a second magnitude wherein transit of the capsule is blocked;
conducting an evaluation of gastrointestinal force based on the data of the first and second magnitude of the magnetic force, the first transit time and the second set of transit time, physical characteristics of the capsule and physical characteristics of gastric contents.

The invention provides a gastrointestinal motility measurement system based on a gastrointestinal capsule, which comprises a data acquisition module, a data processing module and a capsule. The data acquisition module and the data processing module are connected by a wired or wireless communication link. The data acquisition module is configured in the capsule, and comprises an ultrasonic distance measuring device or a 3D camera for acquiring a depth map or a point cloud of the inner wall of the digestive tract. The data processing module is used to process the depth map or the point cloud to extract morphological features, including curvature, inner diameter and volume which are used as references for evaluation of gastrointestinal motility.

The invention provides another gastrointestinal motility measurement system, which comprises a control module, a magnetic driving module, a magnetic positioning module and a capsule. The control module, the magnetic driving module and the magnetic positioning module are connected by a communication link. The capsule is provided with a positioning magnet and a driving magnet, which could be a single magnet or two separate magnets. The positioning magnet generates a magnetic field signal, which is detected by the magnetic positioning module obtaining the position and motion data of the capsule in the digestive tract relative to an external coordinate system. The magnetic driving module generates a driving magnetic field, and the driving magnetic field acts on the driving magnet of the capsule to generate a driving magnetic force to drive the capsule to move in the digestive tract. The control module obtains a first position and motion data of the capsule under the action of gastrointestinal motility through a magnetic positioning module; obtains the second position and motion data of the capsule under the joint action of the gastrointestinal motility and the driving magnetic force. The gastrointestinal motility is estimated according to the first and second position and motion data and the driving magnetic force.

Gastrointestinal motility generally refers to the force and frequency of gastrointestinal contraction, relaxation and peristalsis under the action of gastrointestinal muscles. Its function is to make food move and be transmitted, so as to be digested, absorbed and emptied. An intuitive view of the relationship between the morphological characteristics of the digestive tract and the gastrointestinal motility comprises that under the action of the digestive tract muscles, the gastrointestinal peristalsis first produces deformation, including the change of the curvature of the digestive tract and the change of the inner diameter of the digestive tract. The deformation then transfers the force of the digestive tract muscle to the contents of the digestive tract, such as chyme, so as to make the contents of the digestive tract. Second, the digestive tract, like most other tissues in the human body, can be elastic. It is well known that the force on an elastic body is proportional to the deformation of the body under the force. Therefore, there is a close correlation between the morphological changes of the digestive tract and the gastrointestinal motility. On the other hand, there are significant morphological differences in physiology and pathology of gastrointestinal peristalsis. For example, when stenosis, dilation or obstruction occurs, the normal rhythm of contraction and relaxation will change. Through statistical analysis of the data of the morphological characteristics and the changes of the morphological characteristics and the frequencies of the changes of concerned areas of the digestive tract, a model of the morphological and dynamic characteristics of the digestive tract can be obtained, which can be used as a reference for evaluating the gastrointestinal motility. Like curvature and inner diameter, the morphological characteristics of digestive tract also include the change of volume of target areas of gastrointestinal lumen during peristalsis. The change of volume reflects the emptying amount of gastrointestinal peristalsis, which is related to the work done by gastrointestinal muscles and the energy produced.

The characteristic parameters of the digestive tract proposed above by the invention can preferably be acquired by first obtaining the depth map or point cloud of the inner wall of the digestive tract. Then the morphological features are extracted. Specifically, an ultrasonic distance measuring device can be preferably set in the capsule. After the capsule enters the body, the ultrasonic distance measuring device is started to obtain the distance from the capsule to the inner wall surface of the digestive tract. The ultrasonic measurement device can also collect the distance from the capsule to the multi-layer tissue structure of the inner wall of the digestive tract. Ultrasonic ranging mainly uses time difference ranging method. The ultrasonic transmitter emits directional ultrasonic wave and starts timing at the same time of transmitting. The ultrasonic receiver stops timing after receiving the reflected wave. Let V be the propagation velocity of the ultrasonic wave in the medium, T be the time difference between the transmitted wave and the returned wave recorded by the timer, and S be the distance from the transmitting point to the reflecting point

S=V×T/2

Let the capsule be of a sphere shape, the center of which is located at a point in the digestive tract lumen. The sum of the distance from the point to a point on the inner wall of the digestive tract in an arbitrary direction and the distance from the point to a point on the inner wall of the digestive tract in the opposite direction is defined as the directional cavity diameter of the digestive tract in the present invention. The directional cavity diameter is a measurement of the geometric size of the inner wall of the digestive tract by the ultrasonic ranging device, and also includes a pair of sampling points of the depth map of the inner wall of the digestive tract. There are multiple directional cavity diameters passing through any point. The spatial resolution of the depth map or point cloud and the final surface of the inner wall of the digestive tract is determined by the sampling interval, which conforms to the Nyquist law. A plurality of ultrasonic ranging probes can be preferably set in the capsule to form a spherical directional distribution ultrasonic ranging probe array platform including mechanism, circuit and control software, which is used to obtain multi-directional or panoramic depth map or point cloud data. Obviously, the denser the probe array, the more sampling points, and the higher the corresponding cost and circuit power consumption. Or a mechanical rotation device can be set on the platform of a sparse probe array, and it may rotate an angle after one sampling, and then conduct the next sampling. The platform comprises the following characteristics when conducting one measurement: First, all probes are located on a spherical surface; and second, the ranging directions of the two probes of any pair of probes are opposite, and the connecting lines of the ranging directions of the two probes preferably pass through the ball center; and thirdly, the measurements by two probes of a pair are simultaneous or having an time interval, in which the additional measurement error caused by the time interval is preferably less than that of a single probe.

As the capsule is in a transit under the gastrointestinal peristalsis, the depth map or point cloud data from multiple sampling may preferably be matched, registered and fused. In addition to ultrasonic ranging device, 3D camera based on infrared or visible light sensor can also be used to obtain panoramic depth map or point cloud.

With the peristalsis of the alimentary tract, the capsule moves passively and randomly in the alimentary tract, and is finally discharged from the body. A preferred implementation of the invention can use the magnetic field generated by the magnetic control device to drive the capsule with a magnet in it to move in the digestive tract, or hold the capsule to stay in a target area for a measurement in-situ. Another preferred implementation of the invention is for the capsule to work intermittently, which is used to reduce the power consumption of the capsule battery.

### BRIEF DESCRIPTION OF THE FIGURES

FIG. 1 is a schematic diagram of gastric peristalsis.

FIG. 2 is an example of an ultrasonic capsule operation.

### PREFERRED EMBODIMENT

The present invention is further described in detail in combination with the drawings and the embodiments are for the purpose of explaining and not limiting the present invention.

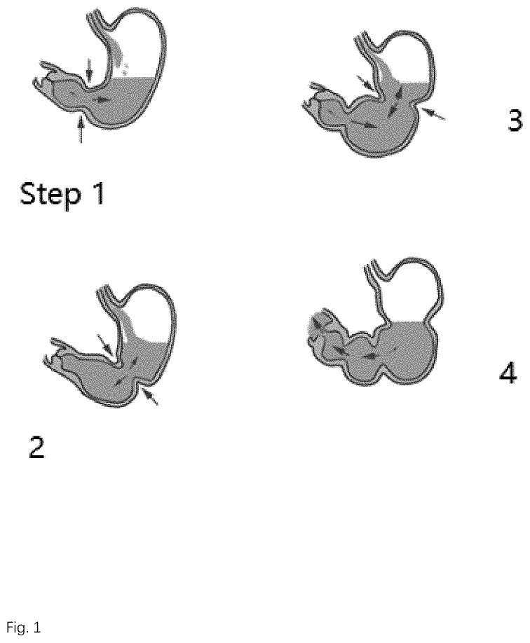

FIG. 1 is a schematic diagram of gastric peristalsis. It shows the changes of gastric wall morphology during peristalsis in the order from 1 to 4.

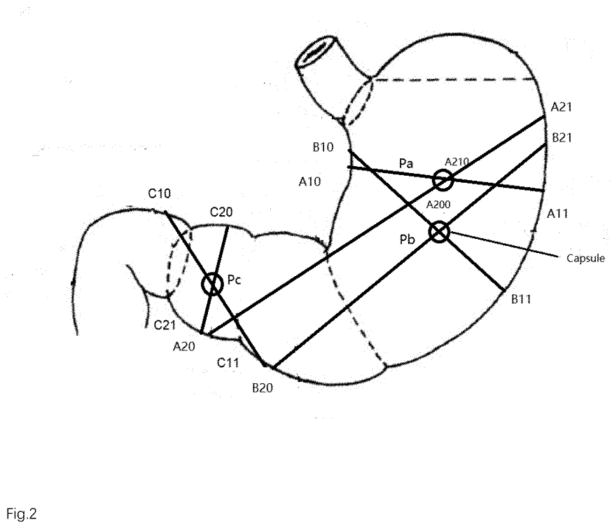

FIG. 2 is an example of an ultrasonic capsule operation. After the capsule enters a subject's body, it can get to a point Pa first. A probe takes a measurement of the distance between a point on an exterior wall of the capsule A210 to a point A21 on the gastric wall along an arbitrary direction of (θ, φ) in a spherical coordinate system with its coordinate origin at Pa, wherein the distance is expressed by |a210, A21|. At the same time, another probe located at A200 on the opposite side of the capsule takes a measure of the distance between A200 to a point A20 on the gastric wall along the opposite direction (−θ, −φ), wherein the distance is expressed by |A200, A20|. Distance of |A210, A21|+|A200, A20|+|A200, A210| is a directional cavity diameter d passing through point Pa. A200 and A210 are the coordinates for two reversely positioned ultrasonic probes. Coordinates (θ, φ, |A210, A21|+½*|A200, A210|) and (−θ, −φ, |A200, A20|+½*|A200, A210|) are a pair of data of ultrasonic depth map obtained by the capsule at point Pa. The collection of the depth data of all points of gastric wall acquired by the capsule at point Pa is the depth map at point Pa. The depth map obtained from different points, such as Pb, Pc, can be matched and fused into a depth map, and then the depth map can be transformed into a point cloud, or each depth map can be transformed into a point cloud, and then the point cloud can be matched and fused. Magnetic positioning may preferably be used to track and mark the pose and position of the capsule as a parameter for depth map or point cloud fusion. The point cloud can be regarded as a sample of the inner surface of digestive tract. Sparse point clouds can be smoothed and denoised by surface fitting to obtain surface data. With the peristalsis of the alimentary canal, the surface data of the inner wall of the whole alimentary canal can be accumulated. Because different parts of the human digestive tract have unique local morphological characteristics and corresponding relationship, the data processing module can recognize the local morphological characteristics of the digestive tract through machine learning. In an example to take a measure of an area of interest, such as a point Pc in FIG. 2, assuming the current position of the capsule being at a point Pa, the magnetic control device can be started to drive the capsule from point Pa to point Pc. When the magnetic positioning device confirms that the capsule has reached point Pc, the system control software of the data processing module starts the ultrasonic ranging device of the capsule to collect data. Furthermore, the data processing module will match the current pose and position data of the capsule collected in real time by magnetic positioning with the pose and position data obtained from analysis of the data of the inner wall of the digestive tract collected by the capsule to ensure the accuracy of the positioning. During a motility test, it may be optimized to minimize the perturbation of the test on the surrounding physiological environment, such as the design of the capsule of a small volume and with a round shape, a sleek shell of the capsule body, and a close density to that of chyme. In a test without intervention, the driving force of the magnetic control equipment can usually be in the zero state. In an intervention test, intervention force can be applied to maintain the capsule in an area of concern, or the capsule motion can be obstructed to measure the gastrointestinal force in the balance. As an embodiment, the capsule is observed at point Pc, near the pylorus. When the magnetic force reaches a first threshold, the transit time of the capsule increases. When the magnetic force reaches a second threshold, the capsule can not be emptied. The peristaltic force of the capsule can then be estimated according to the transit time, the magnitude and direction of the magnetic force, the physical characteristics of the capsule and the physical characteristics of the gastric contents. After obtaining the depth map of the inner wall of digestive tract from the time series collected by the capsule, the data processing module can first convert the depth map into point cloud, and then perform surface fitting. Since the main function of the digestive tract is to move around the food, the direction of food motion can be regarded as the principal axis direction or the principal transit direction of the digestive tract. A statistical average value of a plurality of directional cavity diameters perpendicular to the principal axis at any point in the digestive tract can be set as an inner diameter of the digestive tract at that point. According to the surface data and the anatomic characteristics of digestive tract, the path of the principal transit connecting the points in the digestive tract can be estimated. The calculation of curvature of a surface is a classic subject of differential geometry, and there are a large number of algorithms to choose from. For volume calculation, a length-adjustable line segment (L1, L2) can be selected along the direction of the principal transit as a height, where L1 and L2 are the coordinates of the end points. Through L1 and L2, the vertical plane S1 and S2 in the direction of principal transit are made respectively. A closed body surrounded by surface data of plane S1, S2 and the surface of inner wall of digestive tract can be regarded as a volume at point Pc, which can be calculated by integral numerical method. The motion data of the capsule, including displacement, velocity and frequency, can be obtained by magnetic positioning device. The change rate and range of the above gastrointestinal morphological features can be extracted from the time series data, and the frequency characteristics can be correlated with the frequency characteristics of the capsule motion. Different foods or drugs can affect gastrointestinal motility. The above tests can be carried out in food environment such as water, starch and wine.

What is claimed is:

1. A gastrointestinal motility measurement system, comprising a data acquisition module, a data processing module and a capsule; the data acquisition module and the data processing module are configured to be connected by a wired or wireless communication link, wherein the data acquisition module is configured to be set in the capsule to obtain depth data of inner wall of digestive tract, and the data processing module is configured to process the depth data to extract morphological features, including one or more of position, curvature, inner diameter and volume of the digestive tract.

2. The system of claim 1, wherein the morphological features comprise references for assessment of gastrointestinal motility.

3. The system of claim 1, wherein the data acquisition module comprises a plurality of ultrasonic ranging probes or one or more cameras with infrared or visible light sensor, wherein the ultrasonic ranging probes comprise probe pairs, and two probes of each pair are configured to acquire the depth data of the inner wall of digestive tract along two opposite directions.

4. The system of claim 3, wherein the two probes of each pair are configured to range a first distance from one of the two probes to the inner wall of the digestive tract along a first direction and a second distance from another one of the two probes to the inner wall of the digestive tract along an opposite direction to the first direction, wherein data of the first distance and the second distance comprises a pair of depth data in a depth map,

5. The system of claim 1, wherein the data processing module is configured to fuse depth data obtained in multiple points of positions of the capsule referencing data of the multiple points of the positions of the capsule.

6. The system of claim 1, wherein the data processing module is further configured to:
acquire data of surface of the inner wall of the digestive tract based on the depth data;
acquire a principal axis of the digestive tract;
acquire a line segment (L1, L2) along a direction of the principal axis, wherein L1 and L2 are coordinates of endpoints of the line segment;
obtain plane S1 and S2 vertical to the principal axis comprising L1, L2 respectively;
obtain a volume of the digestive tract enclosed by plane S1, S2 and the surface of the inner wall of the digestive tract.

7. The system of claim 1, wherein the capsule comprises a magnet, and a magnetic sensor outside of the capsule is configured to receive magnetic field signal of the magnet to obtain data of position of the capsule.

8. The system of claim 7, further comprising a magnetic control device; the magnetic control device is configured to generate a magnetic field to move the capsule to, or hold the capsule stay in a target area for a measurement of the morphological features of the target area.

9. An ultrasonic capsule, the capsule comprises a plurality of pairs of ultrasonic ranging probes, wherein two probes of each pair are configured to acquire depth data of inner wall of digestive tract along two opposite directions.

10. The capsule of claim 9, wherein the two probes of each pair are configured to range a first distance from one of the two probes to the inner wall of the digestive tract along a first direction and a second distance from another one of the two probes to the inner wall of the digestive tract along an opposite direction to the first direction, wherein data of the first distance and the second distance comprises a pair of depth data in a depth map.

11. The capsule of claim 9, wherein the plurality of pairs of ultrasonic ranging probes are further configured to obtain panoramic depth map or point cloud of the inner wall of the digestive tract.

12. The capsule of claim 9, wherein the capsule is further configured to:
acquire data of surface of the inner wall of the digestive tract based of the depth data;
acquire a principal axis of the digestive tract;
acquire a line segment (L1, L2) along a direction of the principal axis, wherein L1 and L2 are coordinates of endpoints of the line segment;
obtain plane S1 and S2 vertical to the principal axis comprising L1, L2 respectively;
obtain a volume of the digestive tract enclosed by plane S1, S2 and the surface of the inner wall of the digestive tract.

13-16. (canceled)

17. An ultrasonic capsule, the capsule comprises a plurality of ultrasonic ranging probes configured to obtain data of panoramic depth map or point cloud of inner wall of digestive tract.

18. The capsule of claim 17, wherein the ultrasonic ranging probes comprise probe pairs, and two probes of each pair are configured to acquire depth data of the inner wall of the digestive tract along two opposited directions.

19. The capsule of claim 18, wherein the two probes of each pair are configured to range a first distance from one of the two probes to the inner wall of the digestive tract along a first direction and a second distance from another one of the two probes to the inner wall of the digestive tract along an opposite direction to the first direction, wherein data of the first distance and the second distance comprises a pair of depth data in the panoramic depth map.

20. The capsule of claim 17, wherein the capsule is further configured to:
acquire data of surface of the inner wall of the digestive tract based on the panoramic depth map or point cloud;
acquire a principal axis of the digestive tract;
acquire a line segment (L1, L2) along a direction of the principal axis, wherein L1 and L2 are coordinates of endpoints of the line segment;
obtain plane S1 and S2 vertical to the principal axis comprising L1, L2 respectively;
obtain a volume of the digestive tract enclosed by plane S1, S2 and the surface of the inner wall of the digestive tract.

21. The capsule of claim 18, wherein the capsule is further configured to
obtain a directional cavity diameter of the digestive tract by adding the first distance, the second distance and a third distance between the first and second probes, and obtain an inner diameter of the digestive tract by averaging values of a plurcality of directional cavity diameters.

22. The capsule of claim 10, wherein the capsule is further configured to
obtain a directional cavity diameter of the digestive tract by adding the first distance, the second distance and a third distance between the first and second probes, and obtain an inner diameter of the digestive tract by averaging values of a plurality of directional cavity diameters.

23. The system of claim 3, wherein the capsule is further configured to
obtain a directional cavity diameter of the digestive tract by adding the first distance, the second distance and a third distance between the first and second probes, and obtain an inner diameter of the digestive tract by averaging values of a plurality of directional cavity diameters.
